(19) United States
(12) Patent Application Publication
(10) Pub. No.: US 2025/0229011 A1
(43) Pub. Date: Jul. 17, 2025

(54) THREE-WAY CONNECTING ASSEMBLY AND WEARABLE BREAST PUMP

(71) Applicant: GUANGDONG HORIGEN MOTHER & BABY PRODUCTS CO., LTD., SHANTOU (CN)

(72) Inventors: JIANBIAO CHEN, SHANTOU (CN); YEBIN CAI, SHANTOU (CN); CHANGXIN CHEN, SHANTOU (CN); CHENGBIAO LIN, SHANTOU (CN)

(73) Assignee: GUANGDONG HORIGEN MOTHER & BABY PRODUCTS CO., LTD., SHANTOU (CN)

(21) Appl. No.: 18/815,426

(22) Filed: Aug. 26, 2024

Related U.S. Application Data
(63) Continuation-in-part of application No. PCT/CN2024/071574, filed on Jan. 10, 2024.

(30) Foreign Application Priority Data
Aug. 1, 2023 (CN) 202322045114.5

(51) Int. Cl. A61M 1/06

(52) U.S. Cl. CPC A61M 1/062; A61M 2202/0007; A61M 2202/04; A61M 2205/0216; A61M 2205/075; A61M 2205/3368; A61M 2205/3673; A61M 2210/1007

(57) ABSTRACT

Provided are a three-way connecting assembly and a wearable breast pump. The thrree-way connecting assembly is applied to a wearable breast pump, and includes a three-way connector and a diaphragm holder connected with the three-way connector. The three-way connector is provided with a channel, a milk sucking port, a negative pressure port and a milk outlet. The milk sucking port, the negative pressure port and the milk outlet are spaced apart and are all communicated with the channel. The diaphragm holder includes a hollow plug-in part, a connecting part and an elastic diaphragm. The connecting part connects the hollow plug-in part and the elastic diaphragm. The hollow plug-in part is inserted in the negative pressure port, and the hollow plug-in part and the elastic diaphragm are respectively located at two opposite sides of the three-way connector. The elastic diaphragm is attached to the milk outlet.

### CROSS-REFERENCE OF RELATED APPLICATION

This application claims is a continuation-in-part of International Application No. PCT/CN2024/071574, filed Jan. 10, 2024, which priority to Chinese Patent Application No. 202322045114.5, filed on Aug. 1, 2023, both of which are incorporated herein by reference in their entireties for all purposes.

### TECHNICAL FIELD

The disclosure relates to the technical field of breast pumps, in particular to a three-way connecting assembly and a wearable breast pump.

### BACKGROUND

With the development of science and technology, wearable breast pumps have become popular among breastfeeding women, especially working women. The wearable breast pumps can be worn directly on the user's body or integrated into the user's clothing or accessories, and are used to release the breast milk accumulated in the mammary glands, thus facilitating collection of the breast milk.

The breast pump is generally provided with a valve structure to prevent the breast milk from flowing back and to ensure the suction effect of the breast pump.

However, in the related art, the valve clack of the check valve is connected to the valve seat in an inappropriate manner such that the valve clack would be loosened from the valve seat.

### SUMMARY

In view of this, embodiments of the disclosure provide a three-way connecting assembly and a wearable breast pump.

The embodiments of the disclosure provide the technical solutions as follows.

In an aspect, the embodiments of the disclosure provide a three-way connecting assembly for a wearable breast pump, and the three-way connecting assembly includes a three-way connector and a diaphragm holder. The three-way connector is provided with a channel, a milk sucking port, a negative pressure port and a milk outlet. The milk sucking port, the negative pressure port and the milk outlet are spaced apart, and are all connected with the channel. The diaphragm holder is connected with the three-way connector. The diaphragm holder includes a hollow plug-in part, a connecting part and an elastic diaphragm. The connecting part connects the hollow plug-in part and the elastic diaphragm. The hollow plug-in part is inserted in the negative pressure port, the hollow plug-in part and the elastic diaphragm are respectively located at two opposite sides of the three-way connector, and the elastic diaphragm is attached to the milk outlet.

In another aspect, the embodiments of the disclosure provide a wearable breast pump. The wearable breast pump includes a milk bowl, an airbag, a breast pump flange, a main unit and a three-way connecting assembly. The airbag, the breast pump flange and the main unit are assembed to the milk bowl. The three-way connecting assembly is assembed to the milk bowl, and located between the milk bowl and the breast pump flange. The three-way connecting assembly includes a three-way connector and a diaphragm holder. The three-way connector is provided with a channel, a milk sucking port, a negative pressure port and a milk outlet. The milk sucking port, the negative pressure port and the milk outlet are spaced apart, and are all connected with the channel. The milk sucking port is communicated with the breast pump flange. The diaphragm holder is connected with the three-way connector. The diaphragm holder includes a hollow plug-in part, a connecting part and an elastic diaphragm. The connecting part connects the hollow plug-in part and the elastic diaphragm, the hollow plug-in part is inserted to the negative pressure port, the hollow plug-in part and the elastic diaphragm are respectively located at two opposite sides of the three-way connector, and the elastic diaphragm is attached to the milk outlet. The milk bowl is provided with a vent hole and an airbag mounting groove. The airbag is mounted in the airbag mounting groove and located between the milk bowl and the main unit. The vent hole is communicated with the airbag mounting groove and located in the hollow plug-in part. The milk outlet is communicated with the milk bowl when the elastic diaphragm makes the milk outlet opened.

### BRIEF DESCRIPTION OF THE DRAWINGS

In order to more clearly illustrate technical solutions in the embodiments of the disclosure, drawings to be used in the embodiments are briefly described below. Apparently, the following drawings are merely some embodiments of the disclosure, and those skilled in the art can obtain other drawings according to these drawings without paying any creative effort.

FIG. 1 is a schematic diagram illustrating a structure of a wearable breast pump according to the embodiments of the disclosure.

FIG. 2 is a longitudinal sectional view of the wearable breast pump of FIG. 1.

FIG. 3 is a schematic diagram illustrating a structure of a three-way connecting assembly of FIG. 1.

FIG. 4 is a schematic diagram illustrating structures of a milk bowl and an airbag of the wearable breast pump of FIG. 1.

FIG. 5 is a schematic diagram illustrating a structure of a breast pump flange of the wearable breast pump of FIG. 1.

FIG. 6 is a longitudinal sectional view of the breast pump flange of FIG. 5.

FIG. 7 is a longitudinal sectional view of the breast pump flange according to another embodiment of the disclosure.

### DETAILED DESCRIPTION OF PREFERRED EMBODIMENTS

In order to make those skilled in the art better understand the technical schemes of the embodiments of the disclosure, the technical schemes in the embodiments of the disclosure will be described clearly and comprehensively with reference to the drawings in the embodiments of the disclosure. Apparently, the described embodiments are merely a part rather than all of the embodiments of the disclosure. All other embodiments, obtained by those skilled in the art based on the embodiments in the disclosure without making any creative labor, shall fall within the scope of protection of the disclosure.

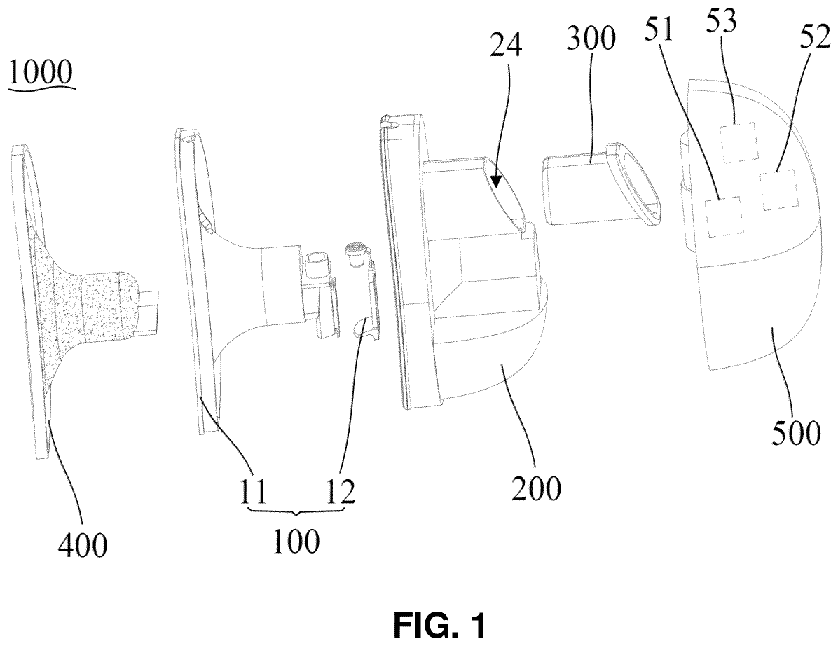

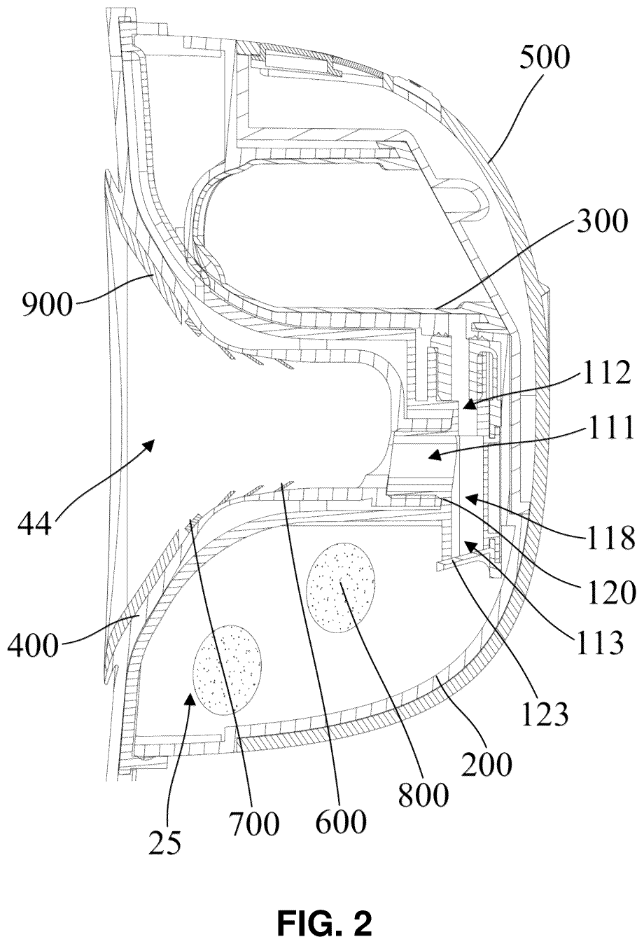

Referring to FIG. 1 and FIG. 2, the embodiments of the disclosure provide a wearable breast pump 1000 that can be worn on a user's breast without holding the breast pump by hand, which is convenient for the user.

In some embodiments of the disclosure, the wearable breast pump 1000 includes a milk bowl 200, an airbag 300, a breast pump flange 400, a main unit 500 and a three-way connecting assembly 100. The airbag 300, the breast pump flange 400, the main unit 500 and the three-way connecting assembly 100 are assembled to the milk bowl 200. The three-way connecting assembly 100 is located between the milk bowl 200 and the breast pump flange 400. The airbag 300 may be a dish-shaped airbag and has a flat shape, and the airbag 300 is located between the milk bowl 200 and the main unit 500.

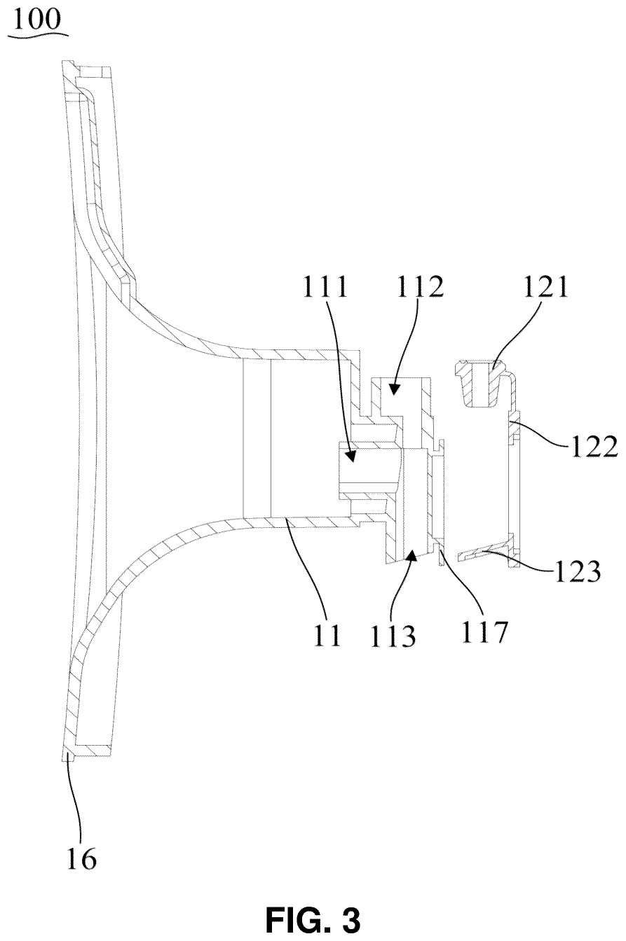

Referring to FIG. 2 and FIG. 3, in some embodiments of the disclosure, the three-way connecting assembly 100 includes a three-way connector 11 and a diaphragm holder 12. The three-way connector 11 is provided with a channel 118, a milk sucking port 111, a negative pressure port 112 and a milk outlet 113. The milk sucking port 111, the negative pressure port 112 and the milk outlet 113 are spaced apart, and are all connected with the channel 118. As such, the three-way connector 11 can meet the needs of the wearable breast pump 1000 for suction, inhalation, and discharge, which is helpful to ensure the normal operation of the wearable breast pump 1000.

In some embodiments, the diaphragm holder 12 is connected with the three-way connector 11. The diaphragm holder 12 includes a hollow plug-in part 121, a connecting part 122 and an elastic diaphragm 123. The connecting part 122 connects the hollow plug-in part 1212 and the elastic diaphragm 123. The hollow plug-in part 121 is inserted in the negative pressure port 112. The hollow plug-in part 121 may be a hollow column, for example, the hollow plug-in part 121 may be a hollow cylinder or a hollow square.

In this way, the diaphragm holder 12 may be connected with the three-way connector 11 via the hollow plug-in part 121, which helps to improve the stability of the connection between the diaphragm holder 12 and the three-way connector 11, and to avoid the situation in which the diaphragm holder 12 comes loose from the three-way connector 11. In addition, it is simple to insert the hollow plug-in part 121 into the negative pressure port 112, which facilitates the installation of the diaphragm holder 12.

In some embodiments, the hollow plug-in part 121 and the elastic diaphragm 123 are respectively located at two opposite sides of the three-way connector 11. In this way, the breast milk would not directly impact the hollow plug-in part 121, which helps to avoid the impact of the breast milk on the hollow plug-in part 121 that would otherwise occurr when the hollow plug-in part 121 and the elastic diaphragm 123 are disposed on the same side of the three-way connector 11, thereby avoiding the loosening of the hollow plug-in part 121 from the three-way member 11 that would otherwise be caused by the impact of the breast milk on the hollow plug-in part 121. In this way, the stability of the connection between the diaphragm holder 12 and the three-way connector 11 is improved.

In some embodiments, the elastic diaphragm 123 is attached to the milk outlet 113. Due to the small weight of the elastic diaphragm 123, the elastic diaphragm 123 is prone to elastic deformation to make the milk outlet 113 opened under the pressure of the breast milk, so as to discharge the breast milk in the three-way connector 11 through the milk outlet 13. This helps to avoid the situation in which the pressure of the breast milk is insufficient to make the milk outlet 113 opened that would be caused due to a heavy weight of the elastic diaphragm 123, thereby ensuring that the three-way connecting assembly 100 discharges the breast milk normally.

In some embodiments, the elastic diaphragm 123 would be attached to the milk outlet 113 under the elastic force, when there is no negative pressure in the three-way connector 11. When the elastic diaphragm 123 makes the milk outlet 113 opened, the milk outlet 113 is communicated with the milk bowl 200, for example, the milk outlet 113 is communicated with a milk storage cavity 26 of the milk bowl 200, so as to store the breast milk discharged from the three-way connector 11.

Exemplarily, when the three-way connecting assembly 100 works, the breast milk may enter the three-way connector 11 from the milk sucking port 111 and flow to the milk outlet 113, and the elastic diaphragm 123 may deform to make the milk outlet 113 opened under the action of the breast milk, thereby allowing the breast milk to flow out of the milk outlet 113. In some embodiments, after the breast milk flows out of the milk outlet 113, the elastic diaphragm 123 may be reset to an initial state under the elastic force, and the elastic diaphragm 123 is reattached to the milk outlet 113, thereby closing the milk outlet 113.

In some embodiments, the connecting part 122 is disposed outside the negative pressure port 112. In this way, it is helpful to avoid the situation that the connecting part 122 hinders the inflation or deflation performed through the negative pressure port 112, and it is helpful to ensure the normal inflation and deflation of the three-way connector 100.

The three-way connector 11 may be further provided with a fixing part 117, and the fixing part 117 may be connected with the connecting part 122, which is helpful to better reduce the loosening of the diaphragm holder 12 from the three-way connector 11. Exemplarily, the fixing part 117 may include a hook, the connecting part 122 may include a slot, and the hook may be snap-fitted in the slot so that the fixing part 117 is connected with the connecting part 122.

In some embodiments, the elastic diaphragm 123 and the milk outlet 113 are arranged along the height direction of the three-way connector 11, and the elastic diaphragm 123 is located below the milk outlet 113. In this way, the elastic diaphragm 123 is easy to elastically deform to make the milk outlet 113 opened under the gravity of the breast milk, which is helpful to improve the efficiency of discharging the breast milk of the three-way connecting assembly 100.

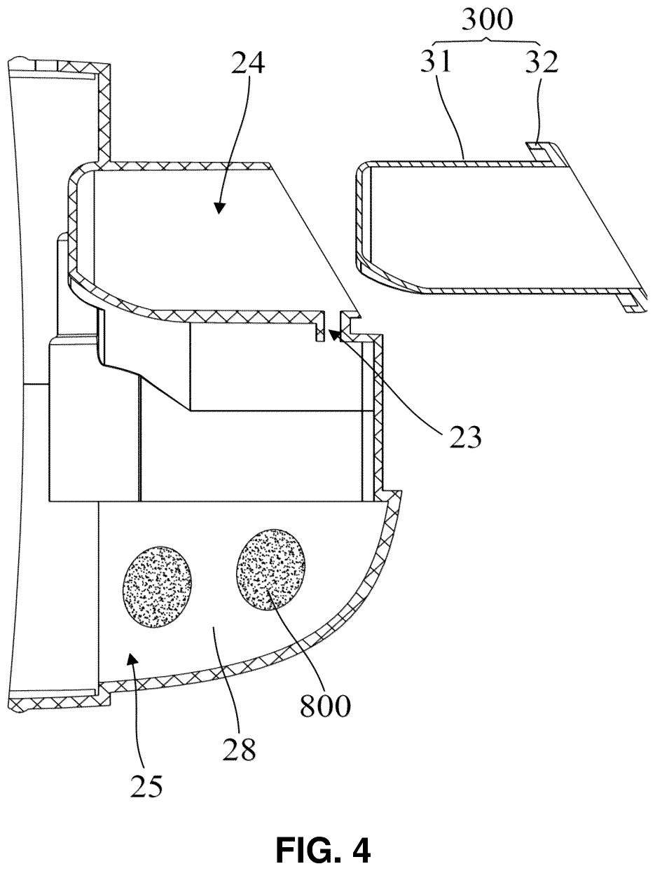

Referring to FIG. 1, FIG. 2 and FIG. 4, in some embodiments, the milk bowl 200 may be provided with a vent hole 23 and an airbag mounting groove 24. The airbag 300 is mounted in the airbag mounting groove 24. The vent hole 23 is communicated with the airbag mounting groove 24, and the vent hole 23 is located in the hollow plug-in part 121.

Because the hollow plug-in part 121 is placed in the negative pressure port 112, the vent hole 23 makes the airbag mounting groove 24 communicated with the negative pressure port 112. The airbag 300 may inhale and deflate to change the negative pressure environment in the three-way connector 11 through the vent hole 23 and the negative pressure port 112, so that the three-way connector 11 maintains therein a suitable negative pressure environment, which helps to enable the milk to flow into the milk sucking port 111.

In some embodiments, the main unit 500 may include a power supply 51, a controller 52 and a vacuum pump 53, the controller 52 is electrically connected with the power supply 51, the controller 52 communicates with the vacuum pump 53 through signal, and the vacuum pump 53 is connected with the airbag 300. For example, the vacuum pump 53 may be connected with the airbag 300 through an air pipe which is located in the vent hole 23.

In this way, the controller 52 may control the vacuum pump 53 to work without manual manipulation, which facilitates the use of the wearable breast pump 1000 by the user.

When it is necessary to pump the breast milk, the breast pump flange 400 is placed on the breast, and the controller 52 controls the vacuum pump 53 to vacuum the airbag 300, so that a negative pressure is generated in the breast pump flange 400 to suck the breast milk.

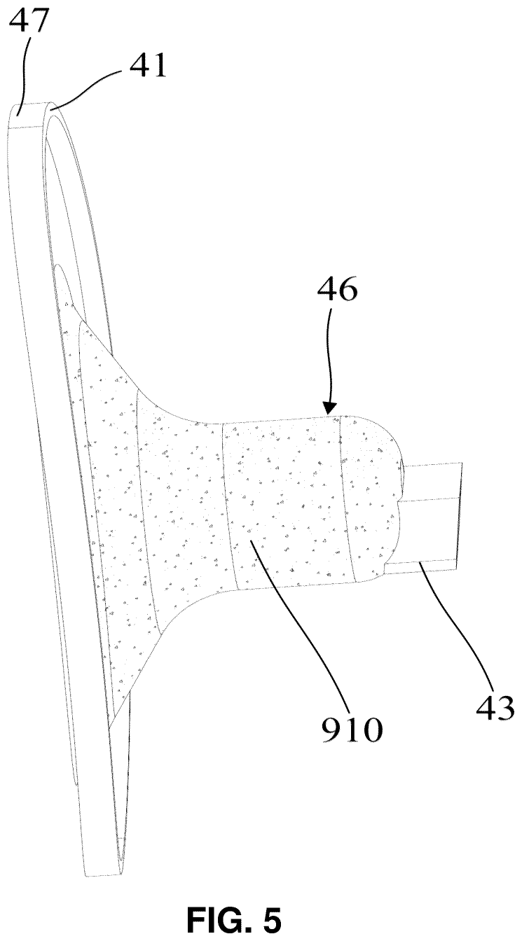

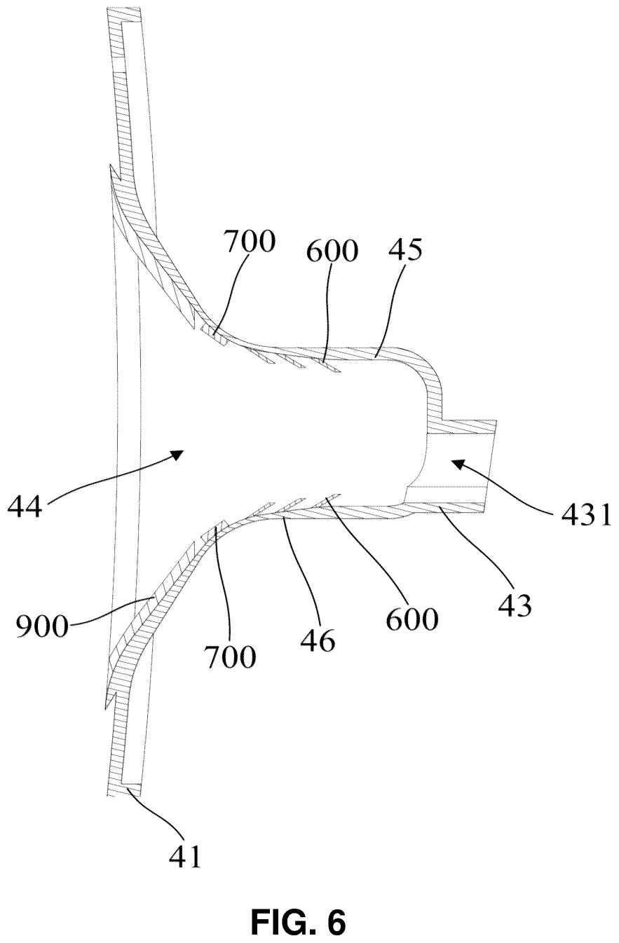

Referring to FIG. 3, FIG. 5 and FIG. 6, in some embodiments, the breast pump flange 400 may be provided with a locating pin 43, the three-way connector 11 is further provided with a plug-in socket 120, the plug-in socket 120 is arranged around an outer peripheral surface of the milk sucking port 111, and the locating pin 43 is inserted into the plug-in socket 120.

In this way, the locating pin 43 helps to locate the connection position between the breast pump flange 400 and the three-way connector 11, thereby enhancing the accuracy of the connection between the breast pump flange 400 and the three-way connector 11, improving the efficiency of assembling the breast pump flange 400 and the three-way connector 11. In addition, it also helps to improve the stability of the connection between the breast pump flange 400 and the three-way connector 11, and helps to reduce disconnection of the breast pump flange 400 with the three-way connector 11.

The milk sucking port 111 is communicated with the breast pump flange 400. For example, the locating pin 43 may be provided with a conneting channel 431 which is communicated with the milk sucking port 111, so that the breast milk in the breast pump flange 400 can flow to the milk sucking port 111 through the conneting channel 431.

In some embodiments, the breast pump flange 400 may be further provided with a locating part 41. The locating part 41 is disposed along a circumferential edge 47 of the breast pump flange 400. The locating part 41 is spaced apart from the locating pin 43. The three-way connector 11 is further provided with a locating connection part 16, and the locating part 41 is connected with the locating connection part 16.

In this way, both of the locating part 41 the locating pin 431 help to locate the connection position between the breast pump flange 400 and the three-way connector 11, so that there is double localization between the breast pump flange 400 and the three-way connector 11. This helps to further enhance the accuracy of the connection between the breast pump flange 400 and the three-way connector 11, further improve the efficiency of assembling the breast pump flange 400 and the three-way connector 11, further improve the stability of the connection between the breast pump flange 400 and the three-way connector 11, and further reduce the disconnection of the breast pump flange 400 from the three-way connector 11.

Exemplarily, the breast pump flange 400 may be trumpet-shaped, and the breast pump flange 400 may be provided with a breast accommodation cavity 44 communicated with the milk sucking port 111. Similarily to the breast pump flange 400, the three-way connector 11 is also trumpet-shaped at a side close to the breast pump flange 400, so that the breast pump flange 400 may be at least partically received in the three-way connector 11. The locating part 41 may be disposed at an edge of the breast pump flange 400 away from the breast accommodation cavity 44.

The locating part 41 may be a rolled-edge flange, and extend inward from the edge of the breast pump flange 400. The locating connection part 16 may be a rolled-edge slot, and extend inward from an edge of the three-way connector 11. In this way, the locating part 41 and the locating connection part 16 help to improve the accuracy of the connection between the breast pump flange 400 and the three-way connector 11, and also help to further improve the stability of the connection between the breast pump flange 400 and the three-way connector 11.

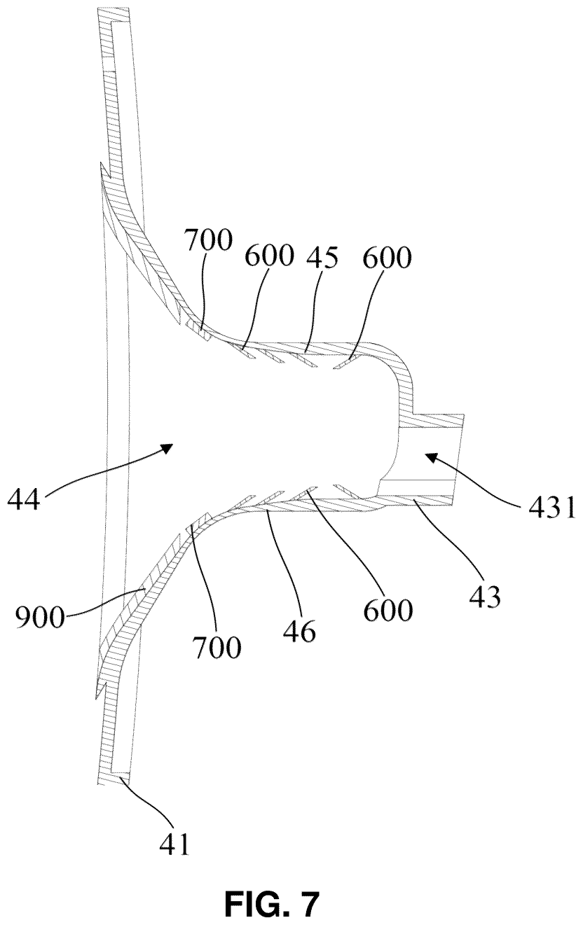

Referring to FIG. 6 and FIG. 7, in some embodiments, the wearable breast pump 1000 further includes multiple anti-overflow plates 600. The multiple anti-overflow plates 600 are arranged at and protrude from an inner wall 45 of the breast pump flange 400, and the multiple anti-overflow plates 600 are spaced along an axial direction of the breast pump flange 400. In this way, the multiple anti-overflow plates 600 can reduce the backflow of the breast milk from the milk suction port 111 to the breast pump flange 400, helping to reduce overflow of the breast milk from the breast accommodation cavity 44, and thus helps to avoid wastage of the breast milk. By providing the multiple anti-overflow plates 600, when a part of the anti-overflow plates 600 close to the milk suction port 111 fail to sufficiently block the breast milk, other anti-overflow plates 600 away from the milk suction port 111 may further block the breast milk, so as to better reduce the overflow of the breast milk from the breast accommodation cavity 44.

In some embodiments, as illustrated in FIG. 6, the part of the anti-overflow plates 600 away from the milk sucking port 111 are tilted in a direction facing towards the milk sucking port 111. Alternatively, each anti-overflow plate 600 is tilted in the direction facing towards the milk sucking port 111. In this way, by arranging of the anti-overflow plates 600 to be tilted in the direction facing towards the milk sucking port 111, it is helpful to guide the breast milk in the breast pump flange 400, so that the breast milk in the breast pump flange 400 can flow to the milk sucking port 111 along a predetermined direction.

In some embodiments, as illustrated in FIG. 7, the part of the anti-overflow plates 600 close to the milk sucking port 111 are tilted in a direction away from the milk sucking port 111. In this way, there may be a large space to receive the breast milk at the position of the breast pump flange 400 near the milk sucking port 111, which helps to improve the anti-overflow effect of the anti-overflow plates 600. Referring to FIG. 2 and FIG. 6, in some embodiments, the wearable breast pump 1000 further includes a vibration massager 700. The vibration massager 700 is assembled in the breast pump flange 400 and arranged close to the anti-overflow plates 600. For example, the vibration massager 700 is disposed on the inner wall 45 of the breast pump flange 400, and is spaced apart from the multiple anti-overflow plates 600. In this way, the vibration massager 700 may be configured to give, through vibration, a massage on the breast to imitate the sucking action of the baby, which is helpful to stimulate the mammary glands to promote the secretion of breast milk and reduce the blockage of the mammary glands. In addition, the vibration massager 700 may transmit vibration to the anti-overflow plates 600, which helps the anti-overflow plates 600 protruding from the inner wall 45 of the breast pump flange 400 to transmit the vibration to the skin surface of the breast, so as to better stimulate the breast and further promote the secretion of breast milk.

In some embodiments, the breast pump flange 400 may be deformable. For example, the breast pump flange 400 may be made of silicone, the silicone breast pump flange can be deformed to better adapt to the user's breast shape, which is helpful to improve the user's comfort in using the wearable breast pump 1000.

In some embodiments, the wearable breast pump 1000 may further include an air cushion 900. The air cushion 900 is attached onto the inner wall 45 of the breast pump flange 400 and disposed at a side of the breast pump flange 400 away from the milk sucking port 111. In this way, the air cushion 900 may be deformed to adapt to different breast shapes of users, so that the air cushion 900 can fit the user's breast, which is helpful to provide a more comfortable sucking experience for users.

The breast pump flange 400 may be further provided with a support for supporting the breast pump flange 400, so that the breast pump flange 400 can maintain a certain shape while deforming, so as to better adapt to the user's breast shape.

Referring to FIG. 5, in some embodiments, the wearable breast pump 1000 may further include an annular heating film 910. The annular heating film 910 is attached onto the breast pump flange 400 and extends along an axial direction of the breast pump flange 400.

In this way, the annular heating film 910 may heat the breast pump flange 400, so that the breast pump flange 400 may be maintained at a proper temperature, which is helpful to improve the comfort of the breast when the user wears the wearable breast pump 1000, to ensure that the breast milk flowing through the breast pump 400 can be maintained at a proper temperature, to reduce the nutrient loss of the breast milk, and to improve the flow speed of the breast milk.

In some embodiments, the annular heating film 910 is located on an outer circumferential surface 46 of the breast pump flange 400. In this way, it is helpful to reduce burn injuries to the user that would be caused due to direct contact between the annular heating film 910 and the user's breast, and it is also helpful to reduce the heat effect of the annular heating film 910 on the vibrating massager 700. The annular heating film 910 may be arranged at a different side from the anti-overflow plates 600 and the vibration massager 700 of the breast pump flange 400, so that the outer circumferential surface 46 of the breast pump 400 may have large area to arrange the annular heating film 910.

Referring to FIG. 2 and FIG. 4, in some embodiments, the wearable breast pump 1000 may further include an electric heating element 800, the milk bowl 200 is provided with a milk storage cavity 25, and the electric heating element 800 is attached to an inner wall 28 of the milk bowl 200 and located in the milk storage cavity 25. The electric heating element 800 may be an electric heating film.

In this way, the electric heating element 800 may heat the inner wall 28 of the milk bowl 200 and the breast milk in the milk storage cavity 25, so that the milk bowl 200 and the brest milk in the milk storage cavity 25 can be maintained at a proper temperature, which is helpful to reduce the nutrient loss of the breast milk and facilitate the user to directly feed the breast milk or store the breast milk.

There may be multiple electric heating elements 800, for example, there may be two, three, four or more electric heating elements 800, which is helpful to increase the heating area of the electric heating elements 800. Alternatively, the electric heating element 800 may cover the inner wall 28 of the milk bowl 200 to ensure that the electric heating element 800 is arranged on the whole inner wall 28 of the milk bowl 200, so as to better improve the heating effect of the electric heating element 800 on the inner wall 28 of the milk bowl 200. This also helps to ensure the uniform temperature of the inner wall 28 of the milk bowl 200, so as to reduce the possibility of deterioration of the breast milk in the milk bowl 200 due to excessive differences in temperature of the breast milk in the milk bowl 200.

In some embodiments, the airbag 300 may include an airbag main body 31 and a locating ring 32 connected with each other, the airbag main body 31 is mounted in the airbag mounting groove 24, and the locating ring 32 is connected with the milk bowl 200 and located outside the airbag mounting groove 24.

In this way, the locating ring 32 helps to locate the connection position between the airbag 300 and the milk bowl 200, improve the accuracy of the connection between the airbag 300 and the milk bowl 200, improve the efficiency of assembling the airbag 300 and the milk bowl 200, improve the stability of the connection between the airbag 300 and the milk bowl 200, and reduce the disconnection of the airbag 300 with the milk bowl 200.

In some embodiments, the milk bowl 200 is transparent, and an outer surface of the milk bowl 200 is provided with a capacity scale. In this way, the milk bowl 200 may be enabled with a milk storage function and a metering function, which helps to facilitate the user to intuitively know the amount of breast milk in the milk bowl 200, and facilitates the user to selectively replenish breast milk into the milk bowl 200 or pour the breast milk out of the milk bowl 200 according to the amount of breast milk.

In the disclosure, unless otherwise expressly specified or limited, the term “assemble” and other terms should be understood broadly. For example, it may mean a fixed connection, a detachable connection or an integral connection, a mechanical connection, a direct connection, an indirect connection through an intermediary, an interaction between two parts, only surface contact, or a connection through surface contact of an intermediary. For those skilled in the art, the specific meanings of the above terms in the embodiments of the disclosure can be understood according to specific situations.

In the disclosure, it is understood that the “first”, “second” etc. are used only for distinguishing descriptions and cannot be understood as specific or special structures. The term “some embodiments” means that a specific feature, structure, material or characteristic described in conjunction with the embodiments or examples is included in at least one embodiment or example of the disclosure. In the embodiments of the disclosure, the schematic descriptions of the above terms are not necessarily aimed at the same embodiment or example. In addition, the specific feature, structure, material or characteristic described herein may be combined in any one or multiple embodiments or examples in a suitable manner. Furthermore, without contradiction, those skilled in the art may combine and incorporate different embodiments or examples and features of the different embodiments or the examples described in the embodiments of the disclosure.

The above embodiments are merely intended to illustrate but not to limit the technical solutions of the disclosure. Although the disclosure has been described in detail with reference to the foregoing embodiments, it can be understood that, those of ordinary skill in the art can modify the technical solutions described in the foregoing embodiments or make equivalent substitutions for some technical features therein. These modifications or substitutions do not drive the essence of the corresponding technical solutions away from the spirit and scope of the technical solutions of the embodiments of the disclosure, and shall fall within the protection scope of the embodiments of the disclosure.

What is claimed is:

1. A three-way connecting assembly for a wearable breast pump, the three-way connecting assembly comprising:
a three-way connector, provided with a channel, a milk sucking port, a negative pressure port and a milk outlet, wherein the milk sucking port, the negative pressure port and the milk outlet are spaced apart and are all connected with the channel; and
a diaphragm holder connected with the three-way connector, wherein the diaphragm holder comprises a hollow plug-in part, a connecting part and an elastic diaphragm, the connecting part connects the hollow plug-in part and the elastic diaphragm, the hollow plug-in part is inserted in the negative pressure port, the hollow plug-in part and the elastic diaphragm are respectively located at two opposite sides of the three-way connector, and the elastic diaphragm is attached to the milk outlet.

2. The three-way connecting assembly as claimed in claim 1, wherein the connecting part is located outside the negative pressure port.

3. The three-way connecting assembly as claimed in claim 1, wherein the elastic diaphragm and the milk outlet are arranged along a height of the three-way connector, and the elastic diaphragm is located below the milk outlet.

4. A wearable breast pump, comprising:
a milk bowl, an airbag, a breast pump flange and a main unit, wherein the airbag, the breast pump flange and the main unit are assembled to the milk bowl; and
a three-way connecting assembly, assembeld to the milk bowl and located between the milk bowl and the breast pump flange;
wherein the three-way connecting assembly comprises a three-way connector and a diaphragm holder connected with the three-way connector;
the three-way connector is provided with a channel, a milk sucking port, a negative pressure port and a milk outlet, the milk sucking port, the negative pressure port and the milk outlet are spaced apart and are all connected with the channel, the milk sucking port is communicated with the breast pump flange;
the diaphragm holder comprises a hollow plug-in part, a connecting part and an elastic diaphragm, the connecting part connects the hollow plug-in part and the elastic diaphragm, the hollow plug-in part is inserted in the negative pressure port, the hollow plug-in part and the elastic diaphragm are respectively located at two opposite sides of the three-way connector, and the elastic diaphragm is attached to the milk outlet; and
the milk bowl is provided with a vent hole and an airbag mounting groove, the airbag is mounted in the airbag mounting groove and located between the milk bowl and the main unit, the vent hole is communicated with the airbag mounting groove and located in the hollow plug-in part, and when the milk outlet is opened through the elastic diaphragm, the milk outlet is communicated with the milk bowl.

5. The wearable breast pump as claimed in claim 4, wherein the connecting part is located outside the negative pressure port.

6. The wearable breast pump as claimed in claim 4, wherein the elastic diaphragm and the milk outlet are arranged along a height of the three-way connector, and the elastic diaphragm is located below the milk outlet.

7. The wearable breast pump as claimed in claim 4, wherein the breast pump flange is provided with a locating pin, the three-way connector is further provided with a plug-in socket, the plug-in socket is arranged around an outer peripheral surface of the milk sucking port, and the locating pin is inserted in the plug-in socket.

8. The wearable breast pump as claimed in claim 7, wherein the breast pump flange is further provided with a locating part, the locating part is disposed along a circumferential edge of the breast pump flange, and the locating part is spaced apart from the locating pin;
the three-way connector is further provided with a locating connection part, and the connecting part is connected with the locating connection part.

9. The wearable breast pump as claimed in claim 4, wherein the wearable breast pump further comprises a plurality of anti-overflow plates, the plurality of anti-overflow plates are arranged at amd protrude from an inner wall of the breast pump flange, and the plurality of anti-overflow plates are spaced along an axial direction of the breast pump flange; and
a part of the anti-overflow plates away from the milk sucking port are titled in a direction facing towards the milk sucking port; or a part of the anti-overflow plates close to the milk sucking port are tilted in a direction away from the milk sucking port; or each of the plurality of anti-overflow plates is titled in a direction facing towards the milk sucking port.

10. The wearable breast pump as claimed in claim 9, further comprising a vibration massager, wherein the vibration massager is assembled in the breast pump flange and arranged close to the anti-overflow plates.

11. The wearable breast pump as claimed in claim 4, further comprising an air cushion, wherein the air cushion is attached onto an inner wall of the breast pump flange and disposed at a side of the breast pump flange away from the milk sucking port.

12. The wearable breast pump as claimed in claim 4, further comprising an annular heating film, wherein the annular heating film is attached to the breast pump flange and extends along an axial direction of the breast pump flange, and the annular heating film is located on an outer circumferntial surface of the breast pump flange.

13. The wearable breast pump as claimed in claim 4, further comprising an electric heating element, wherein the milk bowl is provided with a milk storage cavity, and the electric heating element is attached to an inner wall of the milk bowl and located in the milk storage cavity.

14. The wearable breast pump as claimed in claim 4, wherein the milk bowl is transparent, and an outer surface of the milk bowl is provided with a capacity scale.

15. The wearable breast pump as claimed in claim 4, wherein the airbag comprises an airbag main body and a locating ring connected with each other, the airbag main body ismounting in the airbag mounting groove, and the locating ring is connected with the milk bowl and located outside the airbag mounting groove.

16. The wearable breast pump as claimed in claim 4, wherein the main unit comprises a power supply, a controller and a vacuum pump, the controller is electrically connected with the power supply, the controller is configured to communicate with the vacuum pump through signal, and the vacuum pump is connected with the airbag.
